(19) United States
(12) Patent Application Publication
(10) Pub. No.: US 2009/0105350 A1
(43) Pub. Date: Apr. 23, 2009

(54) Process for the preparation of atovaquone

(75) Inventors: Melvin Anthony Crasto, Navi Mumbai (IN); Ahmed Mubeen Khan, Navi Mumbai (IN)

Correspondence Address: M. CARMEN & ASSOCIATES, PLLC, 170 OLD COUNTRY ROAD, SUITE 400, MINEOLA, NY, 11501, US

(73) Assignee: Glenmark Pharmaceuticals Limited, Mumbai (IN)

(21) Appl. No.: 12/283,249

(22) Filed: Sep. 10, 2008

(30) Foreign Application Priority Data
Sep. 11, 2007 (IN) 1742/MUM/2007

(51) Int. Cl. A61K 31/122; C07C 45/78; A61P 43/00; C07C 49/83

(52) U.S. Cl. 514/682; 568/324; 568/328

(57) ABSTRACT

The present invention provides a process for the preparation of atovaquone exhibiting characteristic peaks (expressed in degrees 2θ±0.2°θ) at approximately one or more of the positions: about 7.0, 9.7, 14.2, 14.8, 17.0, 19.2, 20.4, 22.1, 22.7, 26.9 and 28.7, which comprises: (a) providing a solution comprising atovaquone in an aprotic polar solvent; (b) adding a suitable antisolvent to precipitate atovaquone; and (c) isolating the precipitate.

### PRIORITY

This application claims the benefit under 35 U.S.C. §119 to Indian Provisional Application 1742/MUM/2007, filed on Sep. 11, 2007, and entitled “A PROCESS FOR THE PREPARATION OF ATOVAQUONE”, the contents of which are incorporated by reference herein.

### BACKGROUND OF THE INVENTION

1. Technical Field

The present invention generally relates to a process for the preparation of atovaquone.

2. Description of the Related Art

Atovaquone is chemically described as trans-2-[4-(4-chlorophenyl) cyclohexyl]-3-hydroxy-1,4-naphthalenedione and has the following structural Formula I

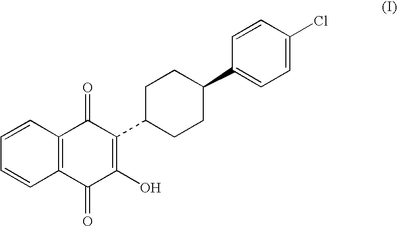

Atovaquone is an antiprotozoal agent and is useful in the treatment of PNEUMOCYSTIS CARINII pneumonia and is commercially available in the market under the brand name MEPRON® as tablets and suspension. U.S. Pat. No. 5,053,432 describes atovaquone and a pharmaceutical composition thereof and exemplifies the crystallization of atovaquone in acetonitrile.

U.S. Patent Application Publication No. 2006/0241311 discloses atovaquone crystalline Form II and Form III and processes for the preparation thereof. However, the process described in U.S. Patent Application Publication No. 2006/0241311 is both expensive and ill-suited to large scale manufacturing.

Polymorphism is the occurrence of different crystalline forms of a single compound and it is a property of some compounds and complexes. Thus, polymorphs are distinct solids sharing the same molecular formula, yet each polymorph may have distinct physical properties. Therefore, a single compound may give rise to a variety of polymorphic forms where each form has different and distinct physical properties, such as different solubility profiles, different melting point temperatures and/or different x-ray diffraction peaks. Since the solubility of each polymorph may vary, identifying the existence of pharmaceutical polymorphs is essential for providing pharmaceuticals with predicable solubility profiles. It is desirable to investigate all solid state forms of a drug, including all polymorphic forms, and to determine the stability, dissolution and flow properties of each polymorphic form. Polymorphic forms of a compound can be distinguished in a laboratory by X-ray diffraction spectroscopy and by other methods such as, infrared spectrometry. Additionally, polymorphic forms of the same drug substance or active pharmaceutical ingredient, can be administered by itself or formulated as a drug product (also known as the final or finished dosage form), and are well known in the pharmaceutical art to affect, for example, the solubility, stability, flowability, tractability and compressibility of drug substances and the safety and efficacy of drug products.

There remains a need to provide an alternative process to the prior art process for making atovaquone, which is cost-effective, feasible and highly reproducible on industrial scale with high yield and purity to provide a stable polymorphic form on a consistent basis.

### SUMMARY OF THE INVENTION

In accordance with one embodiment of the present invention, a process for the preparation of atovaquone exhibiting characteristic peaks (expressed in degrees 2θ±0.2°θ) at approximately one or more of the positions: about 7.0, 9.7, 14.2, 14.8, 17.0, 19.2, 20.4, 22.1, 22.7, 26.9 and 28.7 is provided, which comprises:

(a) providing a solution comprising atovaquone in an aprotic polar solvent;

(b) adding a suitable antisolvent to precipitate atovaquone; and

(c) isolating the precipitate.

In accordance with a second embodiment of the present invention, a solid atovaquone prepared by the process described above and having a purity of at least about 99.5 percent is provided.

In accordance with a third embodiment of the present invention, a pharmaceutical composition is provided comprising atovaquone produced by the above process and a pharmaceutically acceptable carrier or excipient.

### BRIEF DESCRIPTION OF THE DRAWINGS

FIG. 1 is a characteristic powder X-ray diffraction (PXRD) pattern of atovaquone prepared according to Example 2.

FIG. 2 is a characteristic Differential Scanning Calorimetry (DSC) thermogram of atovaquone prepared according to Example 2.

FIG. 3 is a characteristic PXRD pattern of atovaquone prepared according to Example 3.

FIG. 4 is a characteristic DSC thermogram of atovaquone prepared according to Example 3.

FIG. 5 is a characteristic PXRD pattern of a USP reference standard.

FIG. 6 is a characteristic DSC thermogram of a USP reference standard.

### DETAILED DESCRIPTION OF THE PREFERRED EMBODIMENTS

In one embodiment of the present invention there is provided a process for the preparation of atovaquone having a polymorphic form that matches with the USP reference standard of atovaquone as shown in the examples hereinbelow. Generally, the process for the preparation of atovaquone exhibiting characteristic peaks (expressed in degrees 2θ±0.2°θ) at approximately one or more of the positions: about 7.0, 9.7, 14.2, 14.8, 17.0, 19.2, 20.4, 22.1, 22.7, 26.9 and 28.7 involves at least:

(a) providing a solution comprising atovaquone in an aprotic polar solvent;

(b) adding a suitable antisolvent to precipitate atovaquone; and

(c) isolating the precipitate.

In step (a) of the process of the present invention, a solution of atovaquone is provided in an aprotic polar solvent or mixture thereof under suitable conditions. The atovaquone used may be of any indefinite morphology crystalline or may be amorphous or a mixture thereof. Alternatively, the atovaquone used may be crude atovaquone resulting from any synthetic processing step known in the art such as, for example, U.S. Pat. No. 5,053,432, the contents of which are incorporated by reference herein.

The temperature for dissolution of atovaquone will ordinarily range from about 0° C. to about 150° C. and preferably about 60° C. to about 80° C. or reflux temperatures of the solvents used, preferably about 70° C. to about 80° C.

Useful aprotic polar solvents for dissolution of atovaquone include, but are not limited to, aprotic polar solvents such as N,N-dimethyl formamide (DMF), dimethyl sulfoxide (DMSO), dimethylacetamide (DMA), acetonitrile, N-methylpyrrolidinone (NMP), N-methylmorpholine, sulpholane and the like; ketones such as acetone, 2-butanone, methylisobutylketone and the like; and mixtures thereof in various proportions. In one embodiment the aprotic polar solvent is selected from the group consisting of N,N-dimethylformamide (DMF), dimethyl sulfoxide (DMSO), acetonitrile, acetone and N-methylpyrrolidone (NMP) or mixtures thereof. In one preferred embodiment the polar aprotic solvent is DMF. The volume of the solvent used to solubilize atovaquone may range from about 2 to about 20 volumes to the weight of the atovaquone.

If desired, any suspended insoluble matter may be removed by techniques known in the art, e.g., filtration or decantation.

In step (b) of the process of the present invention, a suitable antisolvent is added to the solution to precipitate atovaquone. Useful antisolvents include, but are not limited to, water, hydrocarbon solvents such as n-hexane, n-heptane, cyclohexane and the like; ethers such as dimethylether, diethyl ether, diisopropylether and the like. Mixtures of any of these antisolvents are also contemplated. In one preferred embodiment, water is used as antisolvent. The volume of antisolvent used to precipitate the solid can advantageously range from about 5 to about 100 volumes with reference to volume of the solvent used for solubilizing atovaquone. Generally, the antisolvent is added to the solution at a temperature ranging from about 35° C. to about 100° C.

In one embodiment, the antisolvent is selected from the group consisting of water, n-hexane, n-heptane, dimethylether, diethyl ether, diisopropyl ether and methyl tertiary butyl ether or mixtures thereof. In another embodiment, the antisolvent is water.

In one embodiment, the solvent DMF and the antisolvent water are used in the range of DMF:water (v/v) of about 1:7, and preferably about 1:4.

As used herein, a solvent is any liquid substance capable of dissolving atovaquone.

As used herein, the term “antisolvent” shall be understood to mean a liquid in which a compound is poorly soluble. The addition of an antisolvent to a solvent reduces the solubility of a compound.

Optionally seeding of the desired polymorph is added to the solution of atovaquone to afford the desired polymorph of atovaquone.

If desired, the solution can be cooled by reducing the temperature to about −20° C. to about ambient temperature.

In step (c) of the process of the present invention, the precipitate of atovaquone crystals can then be isolated by techniques known in the art, e.g., filtration, decantation and centrifugation, and then washed and dried to obtain the polymorph form of atovaquone. In one embodiment, the precipitate of atovaquone crystals can be isolated by filtration employing a filtration media of, for example, a celite.

Preferably, the precipitate of atovaquone crystals is isolated by filtering, washing, and drying the solid. Washing is usually done with the same solvent used in the reaction.

The isolated atovaquone is subjected to drying at temperatures from about 30° C. to about 100° C. for a period of about 1 hour to about 15 hours.

The above steps may be repeated to obtain the atovaquone polymorph at a desired purity.

The product may optionally be further dried. Drying can be suitably carried out in a tray dryer, vacuum oven, air oven, fluidized bed drier, spin flash dryer, flash dryer and the like. The temperature for drying can range from about 35° C. to about 90° C. The drying can be carried out for any desired time, such as from about 1 to 20 hours.

In one aspect, the present invention provides solid atovaquone prepared by the process of present invention is further characterized by one or more of the following: (a) having a purity as measured by HPLC of at least about 99.97 percent, and/or (b) having total impurities as measured by HPLC of not more than about 0.05 area percent (%); and/or c) having a water content as measured by Karl Fischer (KF) analysis of not more than about 0.5% w/w; and/or (d) having the following organic volatile impurities below the limit of detection: acetone, acetonitrile, methanol, dichloromethane, 1,4-dioxane, triethyl amine, acetic acid solvents, and having DMF impurity in the range of about 250 ppm to about 670 ppm.

In another aspect, the present invention provides solid atovaquone prepared by the process of present invention and further characterized by one or more of the following: (a) having a purity as measured by HPLC of at least about 99.5 percent; and/or (b) having total impurities as measured by HPLC of not more than about 0.03 area percent; and/or (c) having a water content as measured by KF analysis of not more than about 0.1% w/w; and/or (d) having the following organic volatile impurities below the limit of detection: acetone, acetonitrile, methanol, dichloromethane, 1,4-dioxane, triethyl amine, acetic acid solvents; and having DMF impurity in the range of about 250 ppm to about 670 ppm.

Atovaquone obtained by the process of present invention is characterized by X-ray powder diffraction pattern obtained using a Powder X-ray Diffractometer (Philips X'Pert Pro, PANalytical) with a Cu radiation of λ=1.540598 Å. The measurements were carried out from 2 to 50 degrees with times per step of 50 seconds.

Atovaquone obtained by the process of present invention is further characterized by differential scanning calorimetry (DSC) with the thermogram curve at about 220.27° C. and with an onset temperature curve at about 220.08° C. and an end set temperature curve at about 223.14° C. Measurements of thermal analysis are conducted for the purpose of evaluating the physical and chemical changes that may take place in a heated sample. Thermal reactions can be endothermic (e.g., melting, boiling, sublimation, vaporization, desolvation, solid-solid phase transitions, chemical degradation, etc.) or exothermic (e.g., crystallization, oxidative decomposition, etc.) in nature. The DSC curves presented herein were obtained by using the method which is as as follows:

Approximately 1-5 mg of sample was accurately weighed into an aluminum DSC pan with lid. The sample was then placed into a Mettler Toledo DSC822^{e} equipped with a liquid nitrogen cooling unit and allowed to equilibrate at 30° C. until stable heat flow response was seen. A dry nitrogen purge gas at a flow rate of 50 ml/min was used to produce the inert atmosphere and prevent oxidation of the sample during heating. The sample was scanned from 50 to 250° C. at rate of 10° C./min in a standard Alumina crucibles covered with lids with on hole and resulting heat flow response was measured against temperature.

Preferably, the atovaquone obtained by the process of present invention has a chemical purity of more than about 98%, more preferably more than about 99%, even more preferably more than about 99.5% and most preferably about 99.9% or higher, as measured by HPLC with about 0.1 area % or less total impurities, and in particular a single maximum impurity less than about 0.05 area %.

In addition, the atovaquone obtained by the process of present invention has a low level(s) of residual solvent(s), preferably contains less than about 880 ppm of DMF, and more preferably less than about 600 ppm pf DMF; and acetone, acetonitrile, methanol, dichloromethane, 1,4-dioxane, triethylamine and acetic acid below the detection limit.

In one embodiment, the atovaquone obtained by the process of the present invention is further characterized by residual solvent data as follows: DMF in the range of about 250 ppm to no more than 670 ppm; and acetone, acetonitrile, methanol, dichloromethane, 1,4-dioxane, triethylamine and acetic acid below the detection limit.

Accordingly, D₉₀ particle size of the unformulated atovaquone obtained by the process of the present invention is used as starting material in preparing a pharmaceutical composition generally is less than about 400 microns preferably less than about 200 microns, more preferably less than about 150 microns, still more preferably less than about 50 microns and yet still more preferably less than about 15 microns. Any milling, grinding micronizing or other particle size reduction method known in the art can be used to bring the solid state atovaquone into any desired particle size range as set forth above.

In another embodiment of the present invention, there is provided pharmaceutical compositions containing atovaquone obtained by the process of present invention. Such pharmaceutical compositions may be administered to a mammalian patient in any dosage form, e.g., liquid, powder, elixir, injectable solution, etc. Dosage forms may be adapted for administration to the patient by oral, buccal, parenteral, ophthalmic, rectal and transdermal routes. Oral dosage forms include, but are not limited to, tablets, pills, capsules, troches, sachets, suspensions, powders, lozenges, elixirs and the like. The atovaquone obtained by the process disclosed herein also may be administered as suppositories, ophthalmic ointments and suspensions, and parenteral suspensions, which are administered by other routes. The most preferred route of administration of the atovaquone is oral. The dosage forms may contain the atovaquone as part of a composition. The pharmaceutical compositions may further contain one or more pharmaceutically acceptable excipients.

Capsule dosages will contain the atovaquone which may be coated with gelatin. Tablets and powders may also be coated with an enteric coating. The enteric-coated powder forms may have coatings comprising phthalic acid cellulose acetate, hydroxypropylmethyl cellulose phthalate, polyvinyl alcohol phthalate, carboxymethylethylcellulose, a copolymer of styrene and maleic acid, a copolymer of methacrylic acid and methyl methacrylate, and like materials, and if desired, they may be employed with suitable plasticizers and/or extending agents. A coated tablet may have a coating on the surface of the tablet or may be a tablet comprising a powder or granules with an enteric-coating.

Tableting compositions may have few or many components depending upon the tableting method used, the release rate desired and other factors. For example, the compositions of the present invention may contain diluents such as cellulose-derived materials like powdered cellulose, microcrystalline cellulose, microfine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses; starch; pregelatinized starch; inorganic diluents such calcium carbonate and calcium diphosphate and other diluents known to one of ordinary skill in the art. Yet other suitable diluents include waxes, sugars (e.g. lactose) and sugar alcohols like mannitol and sorbitol, acrylate polymers and copolymers, as well as pectin, dextrin and gelatin.

Other excipients contemplated by the present invention include binders, such as acacia gum, pregelatinized starch, sodium alginate, glucose and other binders used in wet and dry granulation and direct compression tableting processes; disintegrants such as sodium starch glycolate, crospovidone, low-substituted hydroxypropyl cellulose and others; lubricants like magnesium and calcium stearate and sodium stearyl fumarate; flavorings; sweeteners; preservatives; pharmaceutically acceptable dyes and glidants such as silicon dioxide.

Actual dosage levels of the atovaquone disclosed herein may be varied to obtain an amount of atovaquone that is effective to obtain a desired therapeutic response for a particular composition and method of administration for treatment of a mammal. The selected dosage level therefore depends upon such factors as, for example, the desired therapeutic effect, the route of administration, the desired duration of treatment, and other factors. The total daily dose of the atovaquone disclosed herein administered to a host in single or divided dose and can vary widely depending upon a variety of factors including, for example, the body weight, general health, sex, diet, time and route of administration, rates of absorption and excretion, combination with other drugs, the severity of the particular condition being treated, etc.

The process for the preparation of atovaquone of the present invention is simple, eco-friendly and easily scaleable.

The following examples are provided to enable one skilled in the art to practice the invention and are merely illustrative of the invention. The examples should not be read as limiting the scope of the invention as defined in the features and advantages.

### EXAMPLE 1

72 g of atovaquone was dissolved in 345 ml of N,N-dimethyl formamide (DMF) at 70° C. to 75° C. to obtain a clear solution. Next, 2 liters of water was added under stirring to precipitate the solid. The resultant solution was cooled to about 25 to 35° C. followed by filtration of the solid. The filtered solid was washed with water and dried at about 85° C. under vacuum. The material was milled to yield 64 g of atovaquone.

The DSC thermogram showed a sharp endotherm curve at 221.23° C. The x-ray powder diffraction data for this example was performed as described hereinabove and is set forth below in Table 1.

TABLE 1

| Pos. | d-spacing | Rel. Int. |
|---|---|---|
| [°2Th.] | [Å] | [%] |
| 7.0405 | 12.55575 | 54.14 |
| 9.2725 | 9.53783 | 4.62 |
| 9.6908 | 9.12706 | 66.79 |
| 12.7298 | 6.95412 | 1.33 |
| 14.0978 | 6.28228 | 6.37 |
| 14.2720 | 6.20596 | 17.32 |
| 14.8227 | 5.97662 | 21.46 |
| 15.2198 | 5.82157 | 4.64 |
| 17.0996 | 5.18559 | 9.89 |
| 17.9823 | 4.93298 | 1.38 |
| 18.4605 | 4.80626 | 49.39 |
| 18.6132 | 4.76718 | 83.22 |
| 19.2291 | 4.61585 | 100.00 |
| 20.1121 | 4.41515 | 15.89 |
| 20.4737 | 4.33799 | 23.52 |
| 22.1157 | 4.01948 | 38.05 |
| 22.7550 | 3.90798 | 54.46 |
| 23.3375 | 3.81175 | 13.74 |
| 23.7836 | 3.74125 | 2.49 |
| 24.3381 | 3.65726 | 13.15 |
| 25.1781 | 3.53711 | 8.06 |
| 25.6036 | 3.47928 | 1.73 |
| 26.3192 | 3.38629 | 45.13 |
| 26.9027 | 3.31415 | 30.18 |
| 27.2039 | 3.27814 | 9.54 |
| 27.7499 | 3.21486 | 6.57 |
| 28.5539 | 3.12615 | 10.74 |
| 28.7585 | 3.10437 | 13.73 |

### EXAMPLE 2

### Preparation of Atovaquone

33.0 Kg of atovaquone was dissolved in 165 Liters of DMF at about 70° C. to 75° C. to get a clear solution. The hot reaction solution was filtered through sparkler and the sparkler was washed with 2×33 litres of preheated DMF. The resultant filtrate was heated to about 80° C. to 85° C. for about 15 to 30 minutes and 891.0 liters of preheated (70° C.) purified water was added over about 30 to 60 minutes. After completion of the addition, the resultant solution was stirred at about 70 to 75° C. for about 30 minutes. The solution was allowed to cool to about 25 to 30° C. over about 1.5 hours to 2 hours and maintained for 30 minutes. The solid precipitated was centrifuged and the solid was washed with 2×594.0 liters of purified water.

The solid was spin dried for about 60 minutes and then dried in a dryer at about 85 to 90° C. under vacuum for about 12 hours till a loss on drying (LOD) of no more than 0.75% to afford 32.17 kg of atovaquone was obtained.

Purity as measured by HPLC: 99.97 area-%;

Total impurities: 0.03 area %;

Water content by KF: 0.08%;

Organic volatile impurities (O.V.I): acetone, acetonitrile, methanol, dichloromethane, 1,4-dioxane, triethyl amine, and acetic acid solvents are below detection limit. N,N-Dimethyl formamide: 367 ppm.

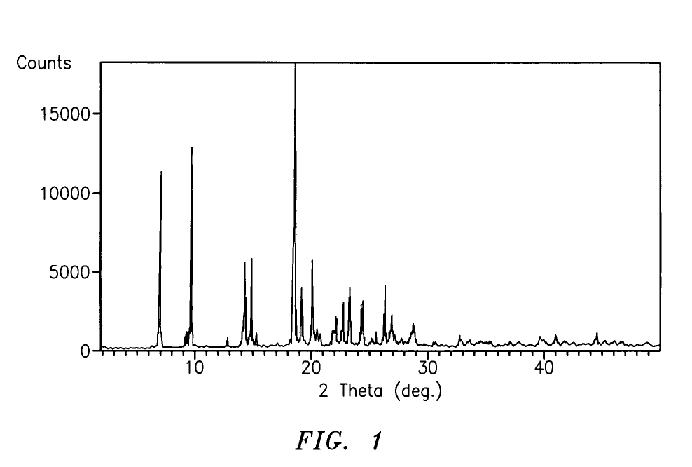

The atovaquone obtained in this examples was characterized by X-ray powder diffraction pattern as shown in FIG. 1 and as set forth in Table 2 below.

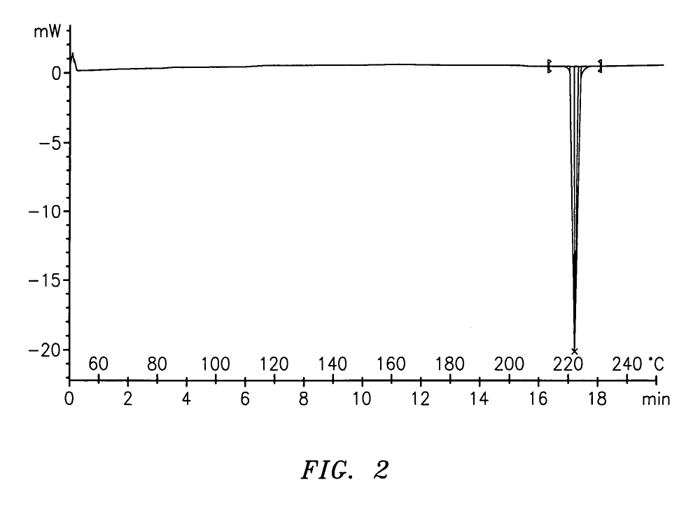

The differential scanning calorimetry (DSC) thermogram showed a sharp endotherm curve at 220.27° C. which is substantially as shown in FIG. 2.

TABLE 2

| Pos. | Rel. Int. |
|---|---|
| [°2Th.] | [%] |
| 7.09 | 64.13 |
| 9.32 | 6.19 |
| 9.74 | 72.78 |
| 12.77 | 3.73 |
| 14.15 | 6.97 |
| 14.32 | 29.76 |
| 14.87 | 31.71 |
| 15.27 | 5.01 |
| 18.66 | 100 |
| 19.27 | 20.13 |
| 20.15 | 30.91 |
| 20.51 | 6.04 |
| 21.95 | 5.74 |
| 22.15 | 10.61 |
| 22.79 | 15.6 |
| 23.37 | 20.28 |
| 24.39 | 15.98 |
| 25.64 | 5.02 |
| 26.35 | 21.71 |
| 26.94 | 10.83 |
| 27.24 | 3.71 |
| 27.76 | 2.94 |
| 28.6 | 5.13 |
| 28.79 | 7.53 |

### EXAMPLE 3

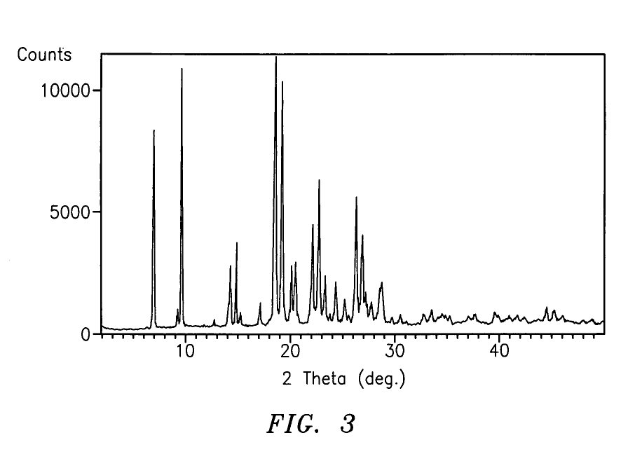

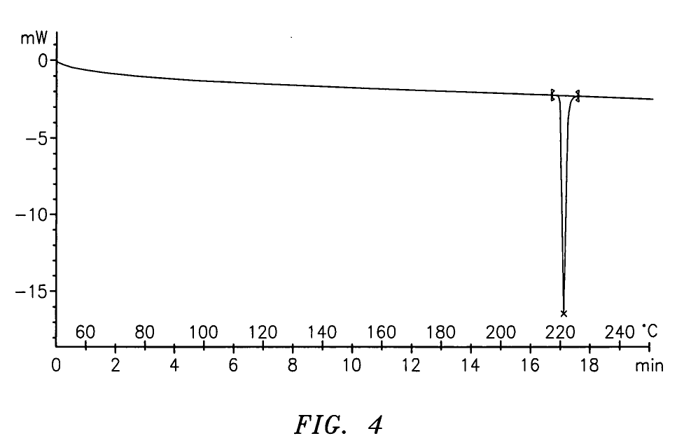

Atovaquone crude dried obtained as per U.S. Pat. No. 4,981,874 was dissolved in 100 volumes of acetonitrile under reflux and the solution was stirred at reflux temperature for 1 hour. The solution was allowed to cool immediately to 0 to 5° C. The slurry obtained was stirred for 4 hrs at this temperature. A solid obtained was filtered off and dried under vacuum at 80° C. for 10 to 12 hrs. The powder X-ray diffraction profile of the solid obtained is shown in FIG. 3 and its details of characteristic spectral lines observed are set forth in Table 3 and compared with that of Form III disclosed in U.S. Patent Application Publication No. 2006/0241311 A1. The DSC thermogram is set forth in FIG. 4 and showed a single sharp endotherm at 220° C.

TABLE 3

| Form III of US | | | | | | |
|---|---|---|---|---|---|---|
| 2006/0241311 | USP Standard Lot # B0F190 | | | Example 3 | | |
| Pos. | Pos. | d-spacing | Rel. Int. | Pos. | d-spacing | Rel. Int. |
| [°2Th.] | [°2Th.] | [Å] | [%] | [°2Th.] | [Å] | [%] |
| 6.99 | 7.0271 | 12.57966 | 57.75 | 7.0311 | 12.57254 | 72.52 |
| | 9.2536 | 9.55721 | 5.6 | 9.2594 | 9.55126 | 6.67 |
| 9.65 | 9.687 | 9.13064 | 70.39 | 9.6874 | 9.13021 | 94.1 |
| 12.67 | 12.688 | 6.97693 | 1.35 | 12.7156 | 6.96187 | 1.96 |
| | | | | 14.0928 | 6.2845 | 8.29 |
| | 14.2629 | 6.20992 | 17.15 | 14.2683 | 6.2076 | 21.85 |
| | 14.8254 | 5.97556 | 22.17 | 14.8173 | 5.97879 | 30.68 |
| | 15.2119 | 5.82458 | 4.31 | 15.2114 | 5.82477 | 5.24 |
| | 17.0951 | 5.18694 | 10.51 | 17.0897 | 5.18858 | 8.68 |
| | 18.4622 | 4.80582 | 73.69 | 18.4388 | 4.81187 | 48.38 |
| | 18.5991 | 4.77077 | 82.73 | 18.6038 | 4.76958 | 100 |
| | 19.2236 | 4.61716 | 100 | 19.215 | 4.61921 | 90.99 |
| 20.07 | 20.0966 | 4.41854 | 16.38 | 20.1045 | 4.41681 | 22.1 |
| 20.65 | 20.4639 | 4.34003 | 26.8 | 20.4563 | 4.34164 | 23.69 |
| 20.99 | | | | | | |
| 22.1 | 22.1107 | 4.02039 | 38.23 | 22.1131 | 4.01996 | 37.09 |
| | 22.7502 | 3.90879 | 52.67 | 22.7415 | 3.91026 | 53.58 |
| | 23.3148 | 3.81541 | 13.59 | 23.3251 | 3.81375 | 18.46 |
| | 23.7573 | 3.74533 | 3.1 | 23.7648 | 3.74416 | 4.14 |
| | 24.3451 | 3.65621 | 12.68 | 24.345 | 3.65623 | 15.92 |
| | 25.1728 | 3.53784 | 7.82 | 25.1647 | 3.53897 | 9.61 |
| | 25.6 | 3.47977 | 2.14 | 25.5858 | 3.48167 | 3.83 |
| | 26.3171 | 3.38655 | 46.13 | 26.3108 | 3.38735 | 47.41 |
| | 26.8738 | 3.31765 | 32.14 | 26.8951 | 3.31507 | 33.72 |
| | 27.2403 | 3.27384 | 9.84 | 27.193 | 3.27943 | 12.32 |
| | 27.7518 | 3.21466 | 6.8 | 27.743 | 3.21565 | 8.41 |
| | 28.5214 | 3.12963 | 13.33 | 28.5476 | 3.12682 | 13.6 |
| | 28.7378 | 3.10656 | 15.84 | 28.7499 | 3.10528 | 15.87 |
| | 29.6993 | 3.00814 | 1.76 | 29.7282 | 3.00528 | 2.78 |
| | 30.5604 | 2.92532 | 2.64 | 30.5403 | 2.9272 | 3.25 |

### EXAMPLE 4

Crude atovaquone (80 g) was added to 1.0 lit acetone and 450 ml methanol. Next, 75 ml triethyl amine was added and stirred and the yellow colored solution changed to a wine red color. The solution was filtered, cooled to 10 to 15° C. and 172 ml acetic acid was added to neutralize the solution to acidic pH. The resultant yellow precipitate was cooled to 0 to 5° C., filtered, and washed with water to yield 73 g of purified material.

Purity as measured by HPLC: >99%.

### EXAMPLE 5

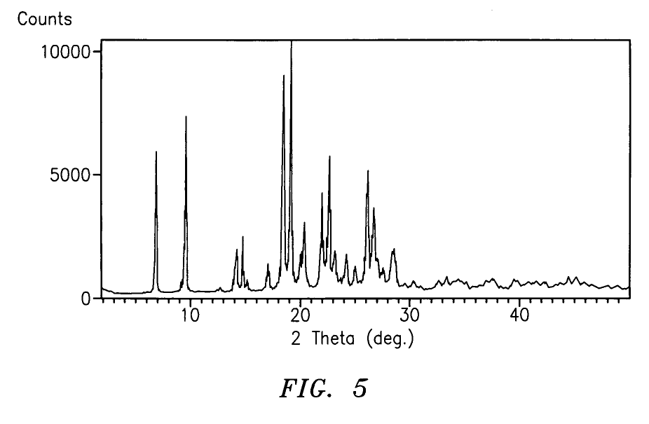

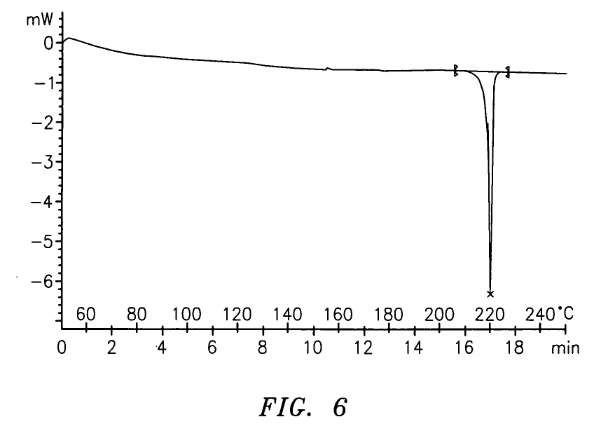

Atovaquone USP reference standard lot # FOB190 released in the market in November-December 2002 was subjected to X-ray powder diffraction analysis and differential scanning calorimetry (DSC) as described above and is shown in FIGS. 5 and 6. The X-ray powder diffraction analysis for the atovaquone USP reference standard lot # FOB190 pxrd is set forth in Table 4 below.

TABLE 4
Peak Table Results for USP Reference Standard Lot # F0B190

| Pos. | d-spacing | Rel. Int. |
|---|---|---|
| [°2Th.] | [Å] | [%] |
| 7.0271 | 12.57966 | 57.75 |
| 9.2536 | 9.55721 | 5.60 |
| 9.6870 | 9.13064 | 70.39 |
| 12.6880 | 6.97693 | 1.35 |
| 14.2629 | 6.20992 | 17.15 |
| 14.8254 | 5.97556 | 22.17 |
| 15.2119 | 5.82458 | 4.31 |
| 17.0951 | 5.18694 | 10.51 |
| 18.4622 | 4.80582 | 73.69 |
| 18.5991 | 4.77077 | 82.73 |
| 19.2236 | 4.61716 | 100.00 |
| 20.0966 | 4.41854 | 16.38 |
| 20.4639 | 4.34003 | 26.80 |
| 22.1107 | 4.02039 | 38.23 |
| 22.7502 | 3.90879 | 52.67 |
| 23.3148 | 3.81541 | 13.59 |
| 23.7573 | 3.74533 | 3.10 |
| 24.3451 | 3.65621 | 12.68 |
| 25.1728 | 3.53784 | 7.82 |
| 25.6000 | 3.47977 | 2.14 |
| 26.3171 | 3.38655 | 46.13 |
| 26.8738 | 3.31765 | 32.14 |
| 27.2403 | 3.27384 | 9.84 |
| 27.7518 | 3.21466 | 6.80 |
| 28.5214 | 3.12963 | 13.33 |
| 28.7378 | 3.10656 | 15.84 |
| 29.6993 | 3.00814 | 1.76 |
| 30.5604 | 2.92532 | 2.64 |
| 31.0824 | 2.87737 | 1.09 |
| 32.7646 | 2.73338 | 3.37 |
| 33.4983 | 2.67518 | 4.76 |
| 34.0363 | 2.63411 | 3.04 |
| 34.8208 | 2.57654 | 3.36 |
| 35.2141 | 2.54866 | 2.58 |
| 36.9745 | 2.43126 | 2.89 |
| 37.6607 | 2.38852 | 3.67 |
| 39.5083 | 2.28098 | 4.05 |
| 40.8482 | 2.20920 | 2.25 |
| 41.6078 | 2.17061 | 2.65 |
| 42.2519 | 2.13900 | 2.21 |
| 44.4530 | 2.03807 | 4.64 |
| 45.1760 | 2.00712 | 4.78 |
| 46.0294 | 1.97187 | 2.57 |
| 47.9429 | 1.89754 | 0.95 |
| 48.8894 | 1.86146 | 1.16 |

The atovaquone USP reference standard lot # FOB190 released in the market in November-December 2002 is believed to show the characteristic peaks of Form III disclosed in U.S. Patent Application Publication No. 2006/0241311.

It will be understood that various modifications may be made to the embodiments disclosed herein. Therefore the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the claims appended hereto.

What is claimed is:

1. A process for the preparation of atovaquone exhibiting characteristic peaks (expressed in degrees 2θ±0.2°θ) at approximately one or more of the positions: about 7.0, 9.7, 14.2, 14.8, 17.0, 19.2, 20.4, 22.1, 22.7, 26.9 and 28.7, the process comprising: (a) providing a solution comprising atovaquone in an aprotic polar solvent; (b) adding a suitable antisolvent to precipitate atovaquone; and (c) isolating the precipitate.

2. The process of claim 1, wherein the aprotic polar solvent is selected from the group consisting of N,N-dimethylformamide (DMF), dimethyl sulfoxide (DMSO), acetonitrile, acetone, N-methylpyrrolidone and mixtures thereof.

3. The process of claim 1, wherein the aprotic polar solvent is DMF.

4. The process of claim 1, wherein the solution of step (a) is prepared at a temperature of about 60° C. to about 80° C.

5. The process of claim 1, wherein the solution of step (a) is filtered using celite.

6. The process of claim 1, wherein the antisolvent is selected from the group consisting of water, n-hexane, n-heptane, dimethylether, diethyl ether, diisopropyl ether, methyl tertiary butyl ether and mixtures thereof.

7. The process of claim 3, wherein the antisolvent is water.

8. The process of claim 1, wherein the antisolvent is water and the antisolvent is added to the solution at a temperature of about 35° C. to about 100° C.

9. The process of claim 1, wherein the isolated atovaquone of step (c) is dried at a temperature of about 30° C. to about 100° C. for a period of about 1 hour to about 15 hours.

10. A solid atovaquone prepared by the process of claim 1, having a purity of at least about 99.5 percent.

11. A solid atovaquone prepared by the process of claim 9, having a purity of at least about 99.97 percent.

12. A solid atovaquone prepared by the process of claim 9, having total impurities of not more than about 0.05 area percent.

13. A solid atovaquone prepared by the process of claim 9, having total impurities of not more than about 0.03 area percent.

14. A solid atovaquone prepared by the process of claim 9, having a water content of not more than about 0.5% w/w.

15. A solid atovaquone prepared by the process of claim 9, having a water content of not more than about 0.1% w/w.

16. A solid atovaquone prepared by the process of claim 9, having an organic volatile impurity of acetone, acetonitrile, methanol, dichloromethane, 1,4-dioxane, triethyl amine, acetic acid solvents below the limit of detection; and DMF present in the range of about 250 ppm to about 670 ppm.

17. A pharmaceutical composition comprising a therapeutically effective amount of a solid atovaquone prepared by the process of claim 1.

18. The pharmaceutical composition of claim 17, further comprising a pharmaceutically acceptable carrier.

19. The pharmaceutical composition of claim 17, further comprising one or more pharmaceutically acceptable excipients.
